(19) United States
(12) Patent Application Publication
(10) Pub. No.: US 2021/0407686 A1
(43) Pub. Date: Dec. 30, 2021

(54) Detecting Early Symptoms And Providing Preventative Healthcare Using Minimally Required But Sufficient Data

(71) Applicant: Koa Health B.V., Barcelona (ES)

(72) Inventors: Teodora Sandra Buda, Madrid (ES); Aleksandar Matic, Lloret de Mar (ES); Mohammed Khwaja, Madrid (ES); Roger Garriga Calleja, Barcelona (ES); Iñaki Estella Aguerri, Barcelona (ES)

(21) Appl. No.: 17/362,886

(22) Filed: Jun. 29, 2021

(30) Foreign Application Priority Data
Jun. 29, 2020 (EP) 20382572.4

(51) Int. Cl. G16H 50/30; G16H 10/60; G16H 50/70; G16H 50/20; G06N 20/00

(52) U.S. Cl. CPC G16H 50/30; G16H 10/60; G16H 40/67; G16H 50/20; G06N 20/00; G16H 50/70

(57) ABSTRACT

A preventative healthcare system calibrates a risk model by assigning weights to attributes for the freshness, completeness and uncertainty of a user's medical information. A risk predictive model is implemented based on the medical information. The risk of a specific health outcome of the user is determined using the risk predictive model, which is calibrated by computing attribute scores for freshness, completeness and uncertainty of the medical information and by assigning weights to the attribute scores. A need-for-data (ND) score is computed using the weighted attribute scores. A need-for-checkup (NC) score is computed using traits of the user. The method determines that new medical information related to the user is needed or that the user needs a checkup based on the ND and NC scores. A prompt is delivered to the user indicating that new medical information related to the user is needed or that the user needs a checkup.

### CROSS REFERENCE TO RELATED APPLICATION

This application is based on and hereby claims the benefit under 35 U.S.C. § 119 from European Patent Application No. EP 20382572.4, filed on Jun. 29, 2020, in the European Patent Office. This application is a continuation-in-part of European Patent Application No. EP 20382572.4, the contents of which are incorporated herein by reference.

### TECHNICAL FIELD

The present invention relates to a computer implemented method, an apparatus and computer programs for early symptom detection and preventative healthcare. The invention allows data requirements to be dynamically set and prompts the patient to supply the minimal required data in order to provide early symptom detection and preventative healthcare by balancing between data requirements defined as completeness, freshness and uncertainty, such that for a specific healthcare outcome and for a specific patient it provides an accurate continuous risk prediction. The feedback loop between data availability, prediction confidence, data requirements, and the known outcome (ground truth) enables individually tailored preventative care and early detection of health related symptoms to be provided using minimally required but sufficient data.

### BACKGROUND

The prior art in the field of processing healthcare-relevant data for enhancing therapy and interventions mainly covers the broad areas: 1) data collection and its maintenance; 2) defining the treatment plan; 3) improving the patient's adherence; and 4) raising alarms in critical moments.

Data collection: Methods in this field focus mainly on how to aggregate and distribute medical information to the healthcare providers. U.S. patent publication US 2011/0225007 focuses on delivering medical information to coaches. A more comprehensive way to handle personal health data is presented in U.S. Pat. No. 7,395,215, which ensures historical data consistency, completeness and availability when needed. In those patent documents, “completeness” refers to gathering all the data from historical records that is located at different healthcare providers. In this disclosure, however, “completeness” refers instead to inferring which data items are needed for a very specific purpose of early detection or prevention of symptoms. The method of the current disclosure does not rely on all of the available information, but rather only on the required information for the specific purpose. The required data is queried if the needed data items do not exist.

Treatment: There are several prior art systems for monitoring mental health symptoms. The most relevant system is disclosed in U.S. Pat. No. 7,540,841, which defines both monitoring mental health symptoms and creating a treatment plan. The treatment plan is defined according to existing medical protocols. Other prior art systems also focus on existing medical protocols. Chinese patent publication CN 101526980A compares the applied therapy plan with the medical protocol. The method of the current disclosure, however, goes beyond the existing protocols and personalizes the protocols not based on the diagnosis (which often can be incorrect) but rather on each individual. Importantly, this does not imply a lack of compliance with existing medical protocols. Rather, modifications of the medical protocol are made for each individual in order to maximize the therapy efficiency (note that effectiveness depends on the adherence) while optimizing healthcare staff engagement. For example, for some patients, check-ups are conducted with a lower frequency (and for some with a higher frequency) with reference to what is defined by the protocol.

Adherence: U.S. application publication 2013/0226617 defines both the treatment plan and the ways to boost adherence to it. A rating score combines individual rating scores for treatment steps of the plan into a single score. Similarly, U.S. Pat. No. 8,566,121 builds user profiles on which interventions for minimizing medical non-adherence are based.

Alerts: This is a broad field and spans from ensuring technical capabilities for delivering alerts when needed to defining how to detect when the right time is to raise an alarm. For instance, U.S. application publications 2017/0098050 and 2006/0058612 focus on the system that delivers health alarms and communication. U.S. Pat. No. 5,576,952 generates alerts based on monitoring units (mainly physiological parameters) and discloses a system and method for comparing against the selected limit parameters.

Other prior art methods are very specific to a certain condition, such as anger and stress. U.S. application publication 2008/0214944 defines how to use health parameters for delivering the therapy. Similarly, U.S. application publication 2011/0245633 using wearable devices to provide diagnosis, treatment and assessment of the treatment effects.

The prior art in the field of data processing to enhance therapy and interventions covers data gathering, treatment plans, adherence and alarms. However, there is no end-to-end system in the prior art that accomplishes the following tasks.
Goes beyond data gathering and maintenance to ensure the quality/certainty, completeness, freshness of the data required for a defined purpose. Rather, the prior art methods focus on general data completeness and gathering the available data collected either through electronic health records or using new technologies.
Treatment plans in the prior art are typically defined “in a vacuum” with no regards to prevention. Instead, the prior art is focused on treatment plans for the diagnosed conditions.
Adherence: Prior art methods in this space provide solutions for making patients adhere to the prescribed therapies. As much as this is important in the medical space, no prior art method describes when adherence in data collection is needed, i.e., when it is critical for patients to adhere to check-ups that (i) result in gathering potentially predictive data of future symptoms and (ii) refine treatment plan with modified healthcare protocols.
Alarms are typically based on detecting when specific parameters are outside of the accepted range and defining ways to signal this to the most appropriate caregiver, family member, or healthcare staff.

Consequently, there is no known end-to-end solution that defines a loop that enables a personalized healthcare protocol and the corresponding preventative care to be administered.

### SUMMARY

A computer implemented method, an apparatus and computer programs for early symptom detection and preventative healthcare are provided. The method involves calibrating a risk prediction model by calculating attribute scores for freshness, completeness and uncertainty of medical and wellness information. Medical and/or wellness information gathered from a user is accessed. A risk predictive model is generated using the accessed medical and/or wellness information. The risk of a specific health outcome of the user is determined using the risk predictive model. A decision control mechanism calibrates the risk predictive model by computing a set of attribute scores defining the freshness, completeness and uncertainty of the medical and/or wellness information. Weights are assigned to the set of attribute scores. A need-for-data (ND) score is computed using the weights and the set of attribute scores. A need-for-checkup (NC) score is computed using various variables of interest and traits of the user. The method determines that new information of the user and/or a health checkup is needed based on the ND score and the NC score.

A method for early symptom detection and preventative healthcare involves calibrating a risk model by assigning weights to attributes for the freshness, completeness and uncertainty of medical information related to a user. Medical information related to the user is accessed from a database. A risk predictive model is implemented based on the medical information related to the user. The risk of a specific health outcome of the user is determined using the risk predictive model. The risk predictive model is calibrated by computing attribute scores for freshness, completeness and uncertainty of the medical information related to the user, and by assigning weights to the attribute scores. A need-for-data (ND) score is computed using the weighted attribute scores. A need-for-checkup (NC) score is computed using the characteristics and traits of the user. The method determines that new medical information related to the user is needed or that the user needs a checkup based on the ND score and the NC score. A prompt is delivered to the user indicating that new medical information related to the user is needed or that the user needs a checkup.

A decision control module determines a usefulness parameter for the prompt by using a machine learning model on the medical information related to the user and the medical information related to individuals similar to the user. The decision control module also determines the usefulness parameter for the prompt by using a deterministic algorithm on the medical information related to the user and on the medical information related to the individuals similar to the user. The ND score is compared to a first threshold, and the usefulness parameter is compared to a second threshold. The prompt to the user indicating that new medical information related to the user is needed is delivered if the ND score is higher than the first threshold and the usefulness parameter is higher than the second threshold.

An electronic system for early symptom detection and preventative healthcare delivers a prompt to the user indicating that new medical information related to the user is needed or that the user needs a checkup. The system includes a database and a processor. The database stores medical information related to the user and medical information related to individuals similar to the user. The processor implements a risk predictive model and a decision control model. The decision control model uses a machine learning model that is built on the database. The processor is configured for assessing the risk of a specific health outcome of the user using the risk predictive model based on the medical information related to the user and the medical information related to individuals similar to the user. The risk predictive model is calibrated by computing attribute scores for the freshness, completeness and uncertainty of the medical information related to the user, and by assigning weights to the attribute scores. The attribute scores for freshness, completeness and uncertainty are computed using the medical information related to the user and the medical information related to the individuals similar to the user. The processor is configured for computing a need-for-data (ND) score using the weighted attribute scores and a need-for-checkup (NC) score using characteristics and traits of the user. The processor is configured for determining that new medical information related to the user is needed or that the user needs a checkup based on the ND score and the NC score. A prompt is delivered to the user indicating that new medical information related to the user is needed or that the user needs a checkup.

The processor is further configured for determining a usefulness parameter for the prompt by using the machine learning model on the medical information related to the user and on the medical information related to the individuals similar to the user. The processor is further configured for comparing the ND score to a first threshold and comparing the usefulness parameter to a second threshold. The prompt to the user indicating that new medical information related to the user is needed is delivered if the ND score is higher than the first threshold and the usefulness parameter is higher than the second threshold.

The processor is further configured for determining a usefulness parameter for the prompt by using a deterministic algorithm on the medical information related to the user and on the medical information related to the individuals similar to the user. The processor is further configured for comparing the NC score to a first threshold and comparing the usefulness parameter to a second threshold. The prompt to the user indicating that the user needs a checkup is delivered if the NC score is higher than the first threshold and the usefulness parameter is higher than the second threshold.

Other embodiments and advantages are described in the detailed description below. This summary does not purport to define the invention. The invention is defined by the claims.

### BRIEF DESCRIPTION OF THE DRAWING

The accompanying drawings, where like numerals indicate like components, illustrate embodiments of the invention.

FIG. 1 schematically illustrates the modules and elements used by the novel system for early symptom detection and preventative healthcare.

FIG. 2 schematically illustrates the modules and elements of another embodiment of the novel system for generating a risk predictive model.

FIG. 3 illustrates the sensors and data sources used by the novel system to gather the medical and/or wellness information of a user.

FIG. 4 schematically illustrates an embodiment of the novel method.

FIG. 5 schematically illustrates how the novel system balances the data requirements for achieving defined levels of accuracy and confidence.

FIG. 6 schematically illustrates another embodiment of the novel system.

FIG. 7 schematically illustrates yet another embodiment of the novel system.

FIG. 8 is a table of perceived wellbeing reported by five individuals and the associated light level and noise level experienced by the individuals.

FIG. 9 is a table of reported wellbeing scores and the associated light and noise levels in which the wellbeing scores have a lower uncertainty.

### DETAILED DESCRIPTION

Reference will now be made in detail to some embodiments of the invention, examples of which are illustrated in the accompanying drawings.

Providing the right intervention at the right time based on continuously acquired information about a user lies at the core of the novel symptom detection and preventative healthcare system. Preventative services represent the holy grail of healthcare, both for healthy individuals (to prevent negative health outcomes in the future) and for patients already diagnosed in order to mitigate and/or prevent the development of their symptoms. In order to do that, one of the critical pre-requisites is to continuously monitor reliable and sufficient information about users and to identify the correct moment to intervene with a necessary check-up or preventative action. The novel method is a solution for how to ensure the acquisition of reliable (minimizing uncertainty), sufficient (complete for the required purpose), and fresh (at least minimally updated for the required purpose) information.

A computer implemented method for early symptom detection and preventative healthcare involves accessing medical and/or wellness information gathered from a user. The medical and/or wellness information is gathered by different data sources, such as medical devices, electronic health records, mobile phones, wearable devices, biochemical and/or genetic tests, diaries, questionnaires and/or surveys. A risk predictive model is generated using the accessed medical and/or wellness information. The risk of a specific health outcome is determined using the risk predictive model. Based on a result of the determined risk, a decision control mechanism calibrates the risk predictive model by computing a set of attribute scores that define freshness, sufficiency and reliability of the accessed medical and/or wellness information. The set of attribute scores is computed using the accessed medical and/or wellness information and the medical and/or wellness information gathered from similar users. Weights are assigned to the computed set of attribute scores. A need-for-data (ND) score is computed using the weights and the computed set of attribute scores. A need-for-checkup (NC) score is computed using different variables of interest and traits of the user. The method determines that new information of the user and/or a health check-up is needed based on the result of the ND and NC scores.

In an embodiment, the medical and/or wellness information, the computed set of attribute scores and the computed ND and NC scores are stored in a database.

In an embodiment, the decision control mechanism determines a usefulness parameter for a prompt to the user by implementing a probabilistic or deterministic algorithm on the data stored in the database. In some embodiments, the algorithm is a probabilistic algorithm based on a machine learning model.

In an embodiment, a first alert mechanism is further implemented by comparing the computed ND score with a first threshold. The usefulness parameter is then compared with a third threshold. A first alert for new medical and/or wellness information is provided if the computed ND score and the usefulness parameter are higher or equal than the first and third thresholds.

In an embodiment, a second alert mechanism is also implemented. This can be done by comparing the computed NC score with a second threshold. The usefulness parameter is compared with a third threshold. A second alert is provided for a new health check-up if the computed NC score and the usefulness parameter are higher or equal than the second and third thresholds.

The first and second alerts can be provided to a computing device, such as a mobile phone, a computer, a smartwatch, a tablet, etc. of the user or of another user relative to the user.

The weights can be equal for each of the set of attribute scores, or can be provided based on the level of priority of the set of attribute scores, or can be even learned by a machine learning model from medical and/or wellness information coming from similar users.

In a second aspect, an apparatus provides early symptom detection and preventative healthcare. The apparatus includes one or more processors and a non-transitory computer-readable storage medium comprising instructions, that when executed, control the one or more processors to be configured to access medical and/or wellness information gathered from a user. The medical and/or wellness information is gathered by different data sources. A risk predictive model is determined using the accessed medical and/or wellness information. The risk of a specific health outcome is determined using the risk predictive model. A decision control mechanism is defined that calibrates the risk predictive model by computing a set of attribute scores that define freshness, sufficiency and reliability of the accessed medical and/or wellness information. The set of attribute scores is computed using the medical and/or wellness information and the medical and/or wellness information of similar users. Weights are assigned to the set of attribute scores. An ND score is computed using the weights and the set of attribute scores. An NC score is computed using different variables of interest and traits of the user. The apparatus determines that new information of the user and/or a new health check-up is needed based on the results of the ND and NC scores.

Other embodiments disclosed herein include software programs that perform the novel method. More particularly, a computer program product is one embodiment that has a computer-readable medium including computer program instructions encoded thereon that when executed on a processor cause the processor to perform the operations indicated herein as embodiments of the invention. The present invention provides an end-to-end solution that dynamically optimizes information gathering directed to each individual to enable personalized symptom tracking, prevention and therapy planning. Aspects of the novel method include:
Data collection: the novel method ensures reliable data sources, data security, transfer, privacy preserving, data provenance, a safe and secure data transfer from the data sources. The novel method ensures completeness, freshness, and certainty for each defined use case. For example, the data can be provided from a reliable source and transferred in a privacy preserving manner, but it may not be “fresh enough” for a specific defined use case.
Treatment: The novel method provides a loop of the therapy planning and the frequency (“freshness”), sufficiency (“completeness”) and reliability (“uncertainty”) of information needed for the particular use case. For example, although standard medical protocol typically defines one mammogram per year for a 45-54 year-old women, that 1-year-old mammogram may be outdated if the risk is perceived higher at an individual level for a specific person. In practice, overwriting a medical protocol is done by doctors at an individual patient level when having check-ups. The novel method determined when to overwrite medical protocols given adequate information. The novel method builds on prior methods that describe how technologies can control for and deliver existing medical protocols and define therapies, with the improvement of the loop between ensuring that appropriate data is acquired and used and tracking symptoms and therapy protocols for each individual patient.
Adherence: The novel method provides a nudge mechanisms for incentivizing adherence to the prescribed therapy and medical protocol. The novel method defines “what” is important to adhere to. Very often defining which data is important and which data is not important (or which therapy is preventative and which is not) can optimize the adherence guidelines making it easier to adhere to the prescribed therapy.
Alerts: The novel method provides timely alerts using the loop between data gathering and the symptom monitoring and therapy planning. The novel method focuses not only on detecting important events, but also considers which data is critical to provide an alarm and to signal when there is a risk of inappropriate alarming, such as once specific data with high predictive power is not received an appropriate form.

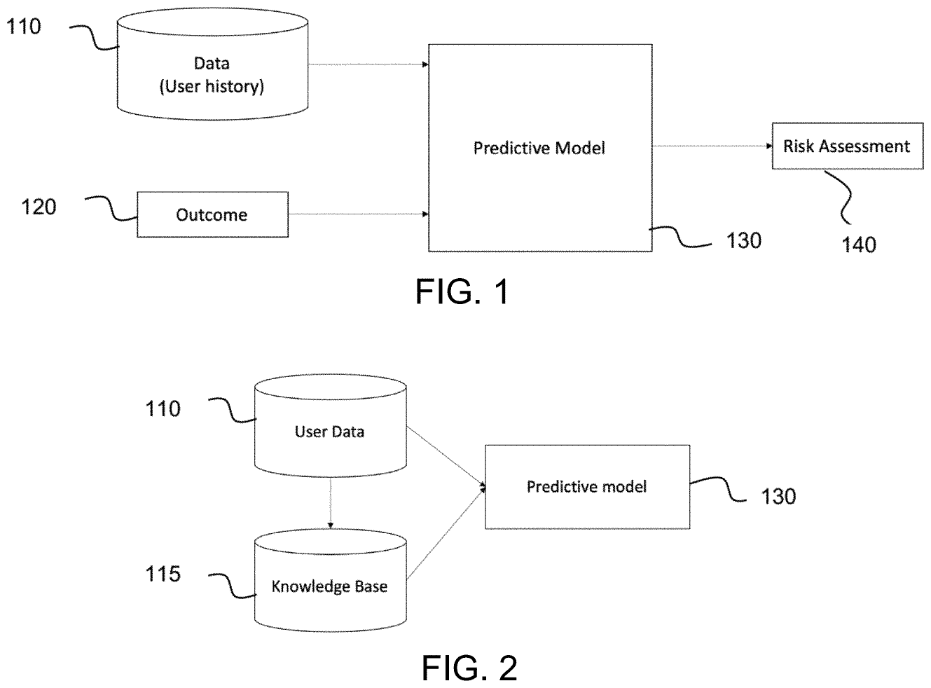

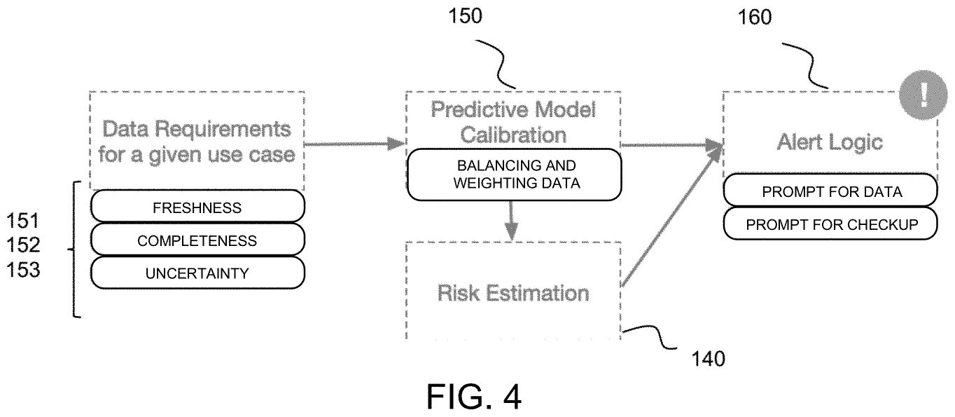

FIG. 1 illustrates a novel system that provides an end-to-end solution delivering prompts when more data, more updated data, or more reliable data 110 is needed for predicting the risk 140 of specific health outcomes. Therefore, it provides two different kinds of alerts: data alerts and check-ups. As shown in FIG. 4, the data requirements (for freshness 151, completeness 152 and uncertainty/reliability 153) are individually set and dynamically updated. The former involves different tailored requirements for each user. For example, an older chronically ill individual at a high risk for a heart-attack would need more updated ECG information than a younger healthy person. The latter involves dynamic updates of the data requirements based on the individual state, but more importantly based on a continuously updated risk prediction model 130 (e.g., with more data 110 gathered) and also in the usefulness of prompts. The novel system provides a confident prediction with less strict data requirements.

Key aspects of the novel system include:

1) The system's data-driven nature allows for the continuous health monitoring of individuals, which enables prevention intervention.

2) The system provides personalized policies for check-ups and interventions. For example, whereas the general recommendation for people 30-40 is an annual check-up for diabetes and hypertension, for one specific person the need for a check-up can be more or less frequent based on the captured parameters and the risk predictive model.

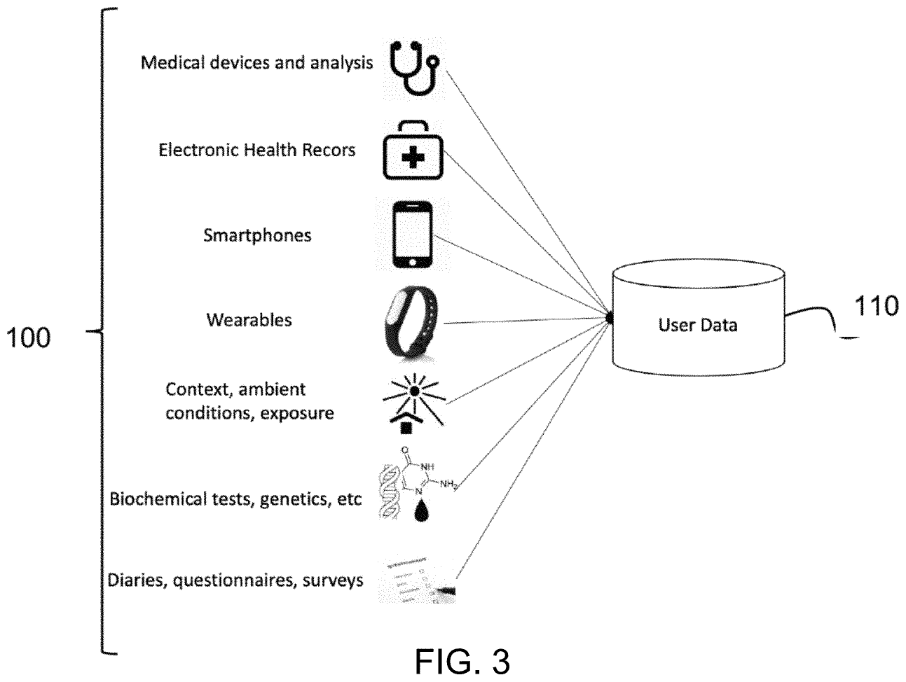

The novel system is adaptable to a range of data sources 100 and health domains as illustrated in FIG. 3. The data sources range from physiological sensing technologies and behavioral modeling from smartphone data to self-reports and parameters captured through typical diagnostic methods, such as physician observations, blood analysis, etc.

The novel system has the following attributes. The system provides the integrated analysis of information 110 about an individual, which considers the relationship between various data inputs and in the context of other individuals and across different use cases. The system provides a continuous evaluation as to whether the existing data 110 about an individual meets the requirements for the risk prediction 140 of a specific outcome. The system provides a mechanism for trading-off data requirements (freshness 151, completeness 152, uncertainty 153) and for maintaining the confidence of the prediction. For example, having a high certainty in one data source can offset the need to have very fresh data from the other sources. The novel method is scalable and continuously assesses the risk as well as the confidence 154 of the risk prediction 140. According to the accuracy confidence 154, the system prompts for more data, a data update, or more certain data and/or prompts for a check-up in order to verify the predicted risk and to update the health outcome.

FIG. 1 schematically illustrates the main concept of the risk assessment of a specific health outcome. The novel system of FIG. 1 considers a continuous or a semi-continuous medical and/or wellness information collection 110 from different data sources 100 (see FIG. 3), which includes “user history” information from the first available information about the user to the present moment. The role of the predictive model 130 of the novel system is to estimate the risk 140 of the user's outcome based on the data available to date. Conceptually, this is similar to medical guidelines built as statistics of risk factors with respect to a patient's static characteristics and dynamic states (health status behaviors over time). For example, a person older than fifty whose family has a history of coronary problems (i.e., has a genetic predisposition as a static personal characteristic) and who has a sedentary lifestyle has a higher risk of heart attack than the average person. Such statistics are made based on the typical medical history information, and the related guidelines are derived. The advent of wearable devices that can measure behavior and physiological signals, advanced medical devices that are continuously improving diagnostics and assessment of a multitude of health parameters, and the increasingly adopted practice of storing more information in electronic health records, can enable personalized and continuous risk assessment at an individual level. In this way, based on aggregated data from a huge number of individuals, the models for individual risk assessment can act in a preventative way and suggest more or less frequent medical check-ups beyond the general guidelines.

Similar to the collection of statistics for patients and healthy individuals in traditional medicine, the predictive model 130 of the novel system also relies on the data from a set of individuals (knowledge base 115) in order to assess the risk 140 of health outcomes for each individual (user data 110), as illustrated in FIG. 2.

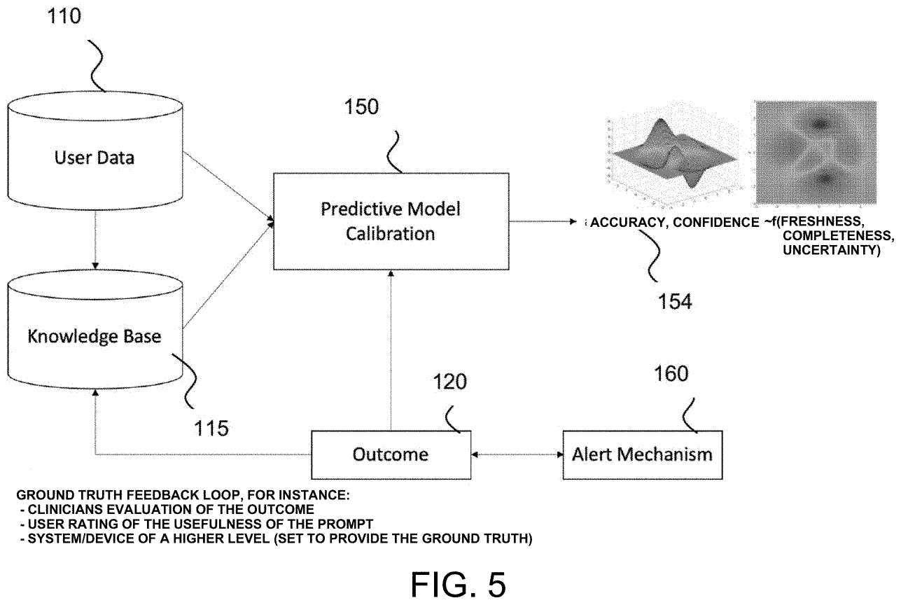

FIG. 4 illustrates an embodiment of the novel method for early symptom detection and preventative healthcare that analyzes the attributes of the input data. The data requirement attributes are freshness 151, completeness 152 and uncertainty 153. The novel method balances and weights the data for freshness, completeness and uncertainty in order to achieve defined levels of accuracy and confidence 154. Then the predictive model 150 of the novel system is calibrated as shown in step 150 of FIG. 5. The system builds risk estimations 140 and prevention healthcare mechanisms at an individual level. The novel system includes an alert mechanism 160 that prompts for a data revision and/or a check-up.

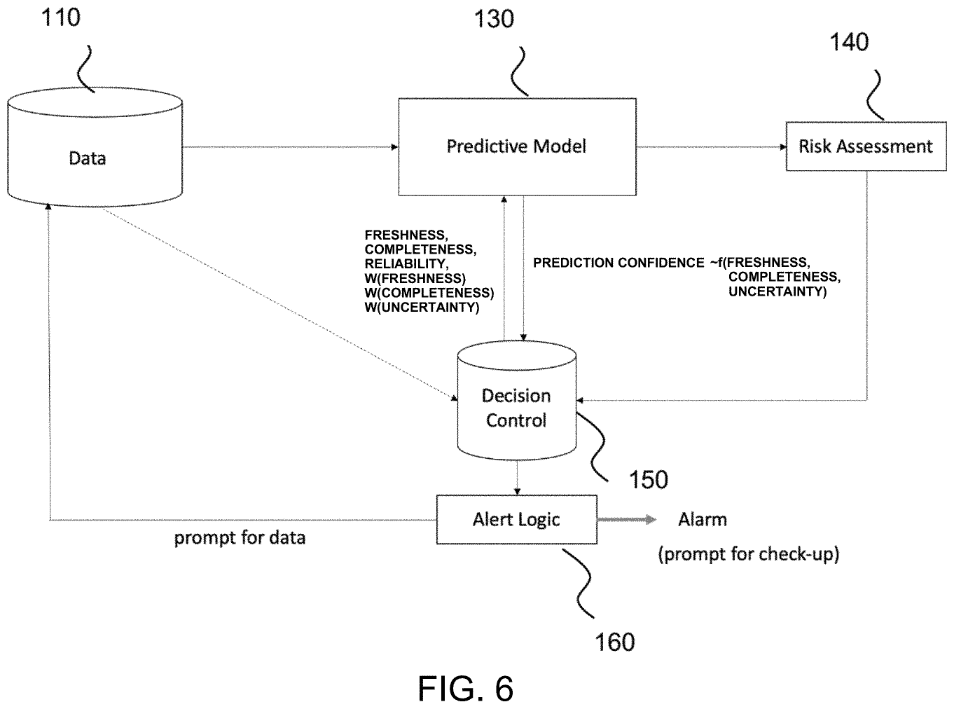

FIG. 6 illustrates another embodiment of the novel system. The data collection and/or storage module 110 is responsible for extracting dynamic information from the user as described above. The dynamic information is used by the predictive model 130 to compute the freshness 151, completeness 152 and uncertainty 153 scores. A decision control module 150 evaluates the freshness 151, completeness 152 and uncertainty 153 scores per user based on the data gathered 110 from the current user and also from similar individuals, both passive and active. This evaluation is performed by the calibration mechanism of the decision control module 150. The calibration mechanism is based on machine learning algorithms that use the prediction confidence 154 to estimate how useful a fresh prompt to the user would be.

FIG. 5 illustrates that the machine learning performed by the predictive model calibration 150 is used to balance among the data requirements of freshness, completeness, and uncertainty. The decision control module 150 iterates various weights that are assigned to freshness, completeness, uncertainty 151-153 in order to find an optimal point for the risk estimation confidence (prediction confidence 154) based on the constraints of the gathered data 110. The weights can be manually overwritten based on the data constraints in order to achieve a new balance within acceptable margins for the remaining data requirements. Then based on the assigned weights for freshness, completeness and uncertainty, module 150 computes the need-for-data score and the need-for-checkup score. Decision control module 150 also predicts the usefulness of a new prompt by using data describing the user's past information. A model in module 150 is trained to predict how useful a future prompt will be for each user. The output obtained from this model is used to inform the alert logic module 160. This prediction also takes into account previous feedback in similar contexts and use cases.

The alert logic module 160 sets the criteria for when the prompt for data or the prompt for a checkup are issued. The alert logic module 160 flags whether the system needs extra data and whether the user needs a checkup at the set moment. In one embodiment, module 160 receives as input the outputs from the decision control module 150, i.e., the scores for freshness 151, completeness 152, uncertainty 153, and their corresponding weights, as well as the prediction of the prompt usefulness. The alert logic module 160 uses the inputs from the decision control module 150 in order to decide whether to issue the prompts based on the need and expected usefulness given the use case.

In one embodiment, the novel system also includes a feedback mechanism module that assesses the data revision and/or the need for a check-up. The feedback can be from a user, a doctor, or a higher level system. The feedback can be implicitly gathered if there is no need to interact with the user or physician. The feedback is explicit whenever a follow-up feedback inquiry is sent to the user or physician. The gathered feedback is fed back to the system in order to improve future risk predictions or future predictions of the data requirement attributes (freshness, completeness, uncertainty).

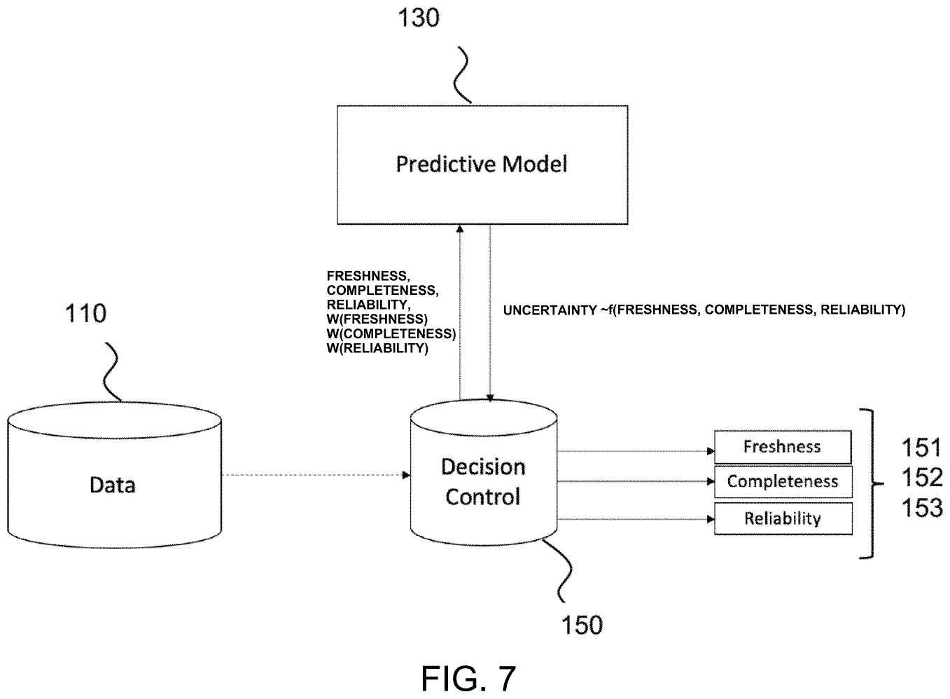

FIG. 7 illustrates another embodiment of the novel system that includes a data module 110, a calibrated predictive model 130, and a decision control module 150. The data collection and/or storage module (user history) 110 contains user history in the form of a collection of past and current records of medical history from the user's electronic health records (EHR). Data module 110 also includes data on self-reported symptoms (e.g., pain, happiness, sleep quality, stress, fever, etc.), self-reported medical history, self-reported personal characteristics (personality, demographics, etc.), passively collected data from smartphones and/or wearables (e.g., heart-rate, galvanic skin response, body temperature, location, phone unlocks, light, speech, ambient noise, phone use, as well as features built on top of the sensor data such as time spent at home, phone use, daily battery consumption), and/or contextual information such as weather, day of the week, and local holiday calendar.

The decision control module 150 is responsible for periodically computing and verifying the data requirements at the periodically user individual level for freshness 151, completeness 152, and uncertainty 153. This is performed in three sub-modules. The freshness sub-module verifies how fresh the user records are and computes a score based on how recently the user added information to the system (both passive and active). The completeness sub-module verifies how complete the user file is. The completeness score represents a percentage of how complete the last information added to the system is. The uncertainty module verifies how uncertain the user file is. The uncertainty score is based both on information provided by other individuals in similar contexts, as well as the user's history in similar contexts. The decision control module 150 also balances the data requirements for achieving defined levels of accuracy and confidence 154, and for predicting the usefulness of a prompt.

Freshness:

The freshness sub-module computes and output the freshness score 151 of the stored data 110 of the user of interest depending on the use case and the relevant outcome 120 ground truth target variable (e.g., wellbeing). The freshness score 151 indicates how updated the information in the system is based on the requirements of the use case (e.g., daily records of time spent at home for depressive patients). Moreover, depending on how static or dynamic in general a variable is, different requirements can be set. For example, if some data remains constant for a long period, this data will be fresh for a longer time, e.g., gender. If data is highly varying, however, the data might need more frequent updates, e.g., body temperature.

A parameter called “age” of information is assessed to quantify the freshness score 151. For a given data component d, a freshness metric is defined in terms of the last timestamp in which the data was collected, t_{last,d,} and the current time, t_{now} with the utility function that depends on both values. For example:

u_{d}t_{last,d},t_{now})=max(0,1−m_{d}×(t_{now}−t_{last,d}))

for m_{d}=1/t_{max, d}

where m_{d} quantifies how fast the utility of the data diminishes over time and t_{max,d} is the maximum time after which the data is fresh, i.e., the utility is 0. Other utility functions can also be used, such as exponential or polynomial.

The freshness score 151 can be provided by data or as the average of all the individual data. Importantly the function parameters can be continuously updated. For instance, for a person suffering from high blood pressure, the measurements related to blood pressure are expected to be weekly, leading to m_{d}= 1/7=0.14. In the case that it has been 10 days with the person not providing this data, the utility function would retrieve a freshness score of: max(0, 1−0.14*(10−0))=max (0, −0.4)=0, where t_{now}=10 and tₗₐₛₜ=0. Thus the utility as given by the age of the parameter would be 0, denoting how fresh the measurement of blood pressure of this person is (the lower, the less fresh the information is).

Completeness:

The completeness sub-module computes the completeness score 152 of the user's stored data 110 based on the user's use case. The completeness score 152 indicates how complete the information in the system is based on how which data is required for a specific outcome prediction. For example, the body temperature and resting state heart rate might be enough to predict an upcoming flu, but only temperature would not be sufficient. In one embodiment, the completeness module implements a Boolean vector for the available data required for the specific task for each of the data components.

Uncertainty:

The gathered data is not always fully accurate. For example, temperature measurements suffer from the sensitivity of the device, self-reports are prone to subjectivity and recall bias, behavioral features captured from a mobile phone might be affected by the sensor limitations, and battery level can impact sampling rate for different sensors which can also stop sending the data. The uncertainty sub-module computes the uncertainty score 153 given the use case requirements and the relevant data, both from the user's past data as well as from data of similar individuals.

This reliability or uncertainty score 153 indicates how unrepresentative the current user's information is compared to the historical user's data, as well as to the data from similar individuals. The uncertainty score 153 indicates the degree of certainty that the captured information is sufficient to predict the outcome, i.e., the associated risk 140. One example of the system inferring a low uncertainty score 153 of the captured information is a self-reported subjective wellbeing score for an extroverted individual who has spent a considerably higher amount of time at home than usual and was not in touch with his friends and family as compared with the past records. As an example, the system would have searched for self-reported subjective wellbeing scores of the other individuals in similar contexts with similar personality traits. Based on the quantified discrepancy with typically reported subjective wellbeing scores, the system computes the uncertainty score, in this example a low uncertainty score.

The interpretation of this score 153 can be twofold. First, the measurements captured through the sensors 100 and active data are incorrect due to faulty circumstances, such as forgetting the phone on the desk at home all day, leaving the phone with the kid, etc. In that case, the uncertainty score 153 would be low. Second, the measurements captured through the sensors 100 and active data are correct, and the user might be experiencing an out of the ordinary situation that is impacting his wellbeing. In this case, the uncertainty score 153 would be high.

The novel system further balances the data requirements between achieving defined levels of accuracy and confidence 154. To do so, dynamic information extracted for the user (freshness, completeness and uncertainty 151-153 and the variables of interest) is used to compute a need-for-data score ND and a need-for-checkup score NC based on the considered use case. The alert logic module 160 uses these scores to decide whether a prompt for data or a prompt for a checkup is activated in the system.

The need-for-data score ND is computed as:

ND=(w₀*freshness)+(w₁*completeness)+(w₂*uncertainty),

where, w₀, w₁ and w₂ are weights that emphasize the value each input variable receives, and Σwᵢ=1. In one embodiment, the weights are equally proportionate, i.e., 0.33 each. In another embodiment, the clinician or patient sets the weights herself based on her priority and use case. In another embodiment, the weights are learned by a machine learning model from data coming from other individuals in a similar use case to understand which of the components are more relevant in general and for that specific moment, considering the variables of interest and the rest of the captured data. This weighting or calibration of the data requirements is crucial for balancing across different data categories depending on the available data, its completeness and uncertainty scores. In one example, a user currently requires his blood pressure, heart rate and breathing rate to be monitored on a weekly basis due to a recent change in his medications. Assuming three weeks of missing measurements on breathing rate, but fresh and complete measurements for blood pressure and heart rate, the alert logic 160 may decide not to raise a prompt for data if the blood pressure and heart rate measurements are sufficient to reach the desired level of accuracy for the use case.

The need-for-checkup score NC is computed as a function of the variables of interest and the traits of the user and is output as the result NC∈{none, low, medium, high}. In one embodiment, there is only one variable of interest, and the clinician sets the three necessary thresholds (between none and low, low and medium, medium and high) on the value of this variable depending on the user's traits. In another embodiment, the thresholds are dependent only on the variable of interest, independently of the user's traits and can be decided by the system or by the physician. In another embodiment with multiple variables of interest vi, each variable is treated independently to obtain a NCᵥᵢ score. Those scores are then converted to the values 0, 1, 2, 3 and added with a weight of importance: NC=Σᵥᵢwᵥᵢ⋅NCᵥᵢ, with Σᵥᵢwᵥᵢ=1. The result is rounded and mapped back to {none, low, medium, high}.

In some embodiments, the usefulness of data revision based on past records can be also predicted. The usefulness determination can be probabilistic (e.g., a machine learning model) or deterministic. After each data revision, ground-truth information about the outcome (in case of checkup) or usefulness of the new data for the model (in case of data prompt) is gathered. The machine learning model uses various input variables including (but not limited to) usefulness ratings of past prompts, psychometric traits (e.g., big five personality, sense of control etc.), past behavior (e.g., relevant activities, visits to the clinician, actions taken upon presentation of a prompt etc.), freshness, completeness and uncertainty given the use case. By using a machine learning regression algorithm, the output of a machine learning model for a user userᵢ is a score, pred_userᵢ that can vary between 1 to 10 based on the scale used for rating the usefulness.

In some embodiments, the machine learning model leverages XGBoost, an ensemble tree-based learning method that is well known for being robust with missing data, and thus complements the calibration component very well.

The alert logic module 160 uses the ND score, the NC score and the predicted usefulness score in order to decide whether a prompt for data or a prompt for a checkup is initiated. The prompt can be sent to a user, an informal or formal caregiver, medical staff, other system, device or similar which is allowed to define a “ground-truth” about the outcome and then inform the feedback mechanism component.

If both the ND score is above a certain threshold T_{ND}, and the predicted usefulness score is higher than a certain threshold T_{pred}_{_{_}}ᵤₛₑᵣ_{ᵢ}, the novel system requires new data to be acquired. The predicted usefulness score is then used to determine whether the user must be prompted directly to take action, or the prompt should go to another person from the user's circle. It may be the case that the data that is missing can only be gathered through a visit to the clinician. In that case, the user would need to go to the clinician to gather that data and then input the data into the system, following the prompt.

If both the NC score is above a certain threshold T_{NC}, and the predicted usefulness score is higher than a certain threshold T_{pred}_{_{_}}ᵤₛₑᵣ_{ᵢ}, the system prompts for a checkup. Moreover, in one embodiment, the prompt for a checkup is a prompt to gather new information at the checkup about the user, about the outcome, or a risk factor. Therefore, the prompt can also be triggered when the uncertainty score 153 is high, or the freshness 151 or completeness 152 score are low, or when the predicted usefulness score is high.

This type of alert targets the user for early detection of symptoms and asks for additional information in case the system detects that the user file is either: deprecated/stale, incomplete, or the uncertainty level detected by the system is high.

The prompt score is general and is used for early detection of symptoms, timely provision of prompts and deciding when new data must be collected. The prompt score can be further grouped into discrete states such a low, medium and high to indicate the severity of the condition, and this state indicates what action the user or clinician will be asked to take. The prompt score can also be binarized, i.e., given a value of 1 or 0 (if the score is above or below a threshold, respectively) to decide when historical information about the user needs to be refreshed.

In an embodiment of the novel system related to mental health, the prompts can be sent to friends or family of the user instead of to the user. The user can define a “friends and family” circle that consists of all the people in the user's life who are important to him or her. The user is always in the center of the circle (first level), followed by the user's closest ally (e.g., partner) in the second level, and followed by the user's best friend in the third level, and so on. The clinician and a close neighbor can be placed in the third or fourth level, based on the user's past behavior. A structure of prompts can be built where for levels of low severity, only the user is prompted to take action. For medium severity conditions, the user and the user's closest ally in the second level are prompted on their respective mobile phones. Thus, there is a follow up done in the physical world that compels the user to take action. In cases of high severity, all members of the circle within the third or fourth levels are informed, so that there is a follow up from all of the user's trusted allies in the physical world to push the user to visit a clinician or to do a checkup.

The feedback mechanism assesses the data revision and/or need for a checkup, such as from a user and a physician or a high level system. The feedback can be reported by the user, a physician, an entity, other device or system considered as “ground-truth”, in order to determine the utility of the data or checkup prompt. In particular, the feedback gathered can be implicit if there is no need to interact with any person or entity, or explicit whenever a follow-up feedback inquiry is sent to the user or physician. In one embodiment, the usefulness of a prompt can be learned by the system based on the actions of the user, such as the user providing fresh updated data in the case of a prompt for data, or attending a checkup in the case of a prompt for checkup.

Furthermore, the feedback gathered is fed back to the system in order to improve future risk predictions and future predictions of the data requirement attributes (freshness 151, completeness 152, uncertainty 153). In particular, the decision control module 150 uses the feedback to calibrate the predictive model 130. The weights used by the predictive model 130 are adjusted to balance the data requirements based on the observed important data for the prompt.

An example of the computation of the uncertainty score 153 is described below.

Case I: Given the ground truth, i.e., an active current wellbeing momentary measurement for an individual i, the past records belonging to the current individual and to individuals with similar traits with a similar active wellbeing measurement are filtered, and the following uncertainty score 153 is computed:

Uncertaintyⁱ(t)=1/n*Σ(Sⁱ_{f}(tₚᵣₑₛₑₙₜ)−1/m*ΣS^{i+s}^{ⁱ}_{f}(tₚₐₛₜ)|)

The score indicates the average absolute difference for each sensor feature Sⁱ_{f} with f from 1 to n, between the current sensor feature measurement Sⁱ_{f}(tₚᵣₑₛₑₙₜ) and the mean of past sensor feature measurements S^{i+s}^{ⁱ}_{f}(tₚₐₛₜ), both from this individual i and individuals with similar traits Sⁱ, with i from 1 to m, when reporting a similar active wellbeing measurement.

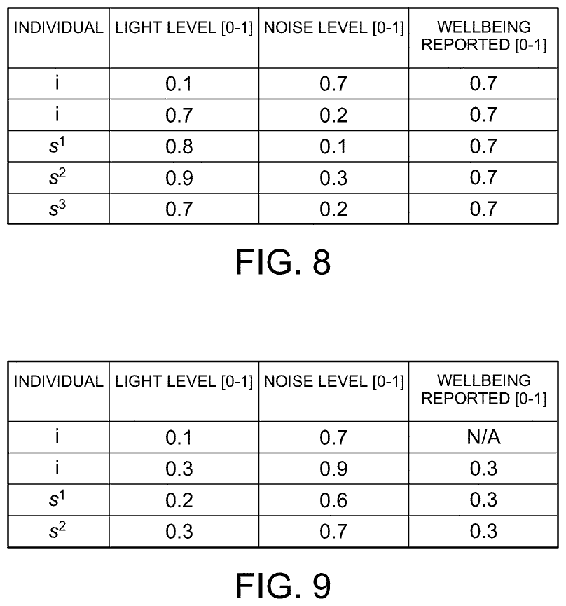

In this particular case, the uncertainty score 153 corresponds to the first of the two interpretations described above in which measurements captured by the sensors 100 are incorrect due to faulty circumstances. In this case, the light level and the noise level are sensor-based features with the measurements listed in the table of FIG. 8. In this case, the decision control module 150 searches for records with similar wellbeing reported. This leads to filtering records of the individual i and similar individuals with similar reported wellbeing, producing the table of FIG. 8.

The uncertainty score 153 corresponding to the data of FIG. 8 would be: ½[|0.1−(0.7+0.8+0.9+0.7)*¼|]+[|0.7−(0.2+0.1+0.3+0.2)*¼|]=½*[0.675+0.5]=0.59. This corresponds to a high uncertainty score 153, meaning that most likely the measurements from the sensors were faulty.

Case II: When the ground truth information is missing, the decision control module 150 searches for records of individuals with similar traits, in similar contexts, and with matching passive sensor measurements. The decision control module 150 computes the mean wellbeing target score, and based on that retrieves records of the current individual from the past where the user reported similar wellbeing scores. Based on these records, module 150 then computes the uncertainty score 153 based on the formula above.

In the majority of situations, the uncertainty score 153 in this case would be low, based on the assumption that there is a vast literature on predicting wellbeing scores from passive sensing data.

In this particular case, the uncertainty score 153 corresponds to the second of the two interpretations described above in which the measurements captured through the sensors 100 are correct, and the user is experiencing an out of the ordinary situation. In this case, the light level and the noise level are sensor-based features, and the measurements are listed in the table of FIG. 9. The first row represents the current row under analysis.

The first step involves retrieving records with similar sensor measurements for the sensors recorded of the same individual i and similar individuals, resulting in rows 2-4. The second step involves computing the average wellbeing in the records, resulting in a wellbeing of 0.3. The third step involves filtering records of the individual i of similar wellbeing, finally producing the table of FIG. 9. The uncertainty score 153 would be: ½*[|0.1−(0.3+0.2+0.3)*⅓|]*[|0.7−(0.9+0.6+0.7)*⅓|]=½*[0.16+0.03]=0.095

This corresponds to a low uncertainty score 153, meaning that indeed the user might be exhibiting an unusual behavior.

Various aspects of the proposed method, as described herein, may be embodied in programming. Program aspects of the technology may be thought of as “products” or “articles of manufacture” typically in the form of executable code and/or associated data that is carried on or embodied in a type of machine readable medium. Tangible nontransitory “storage” type media include any or all of the memory or other storage for the computers, processors, or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, from a management server or host computer of a scheduling system into the hardware platform(s) of a computing environment or other system implementing a computing environment or similar functionalities in connection with data processing. Thus, another type of media that may bear the software elements includes optical, electrical and electromagnetic waves, such as used across physical interfaces between local devices, through wired and optical landline networks and over various airlinks. The physical elements that carry such waves, such as wired or wireless links, optical links or the like, also may be considered as media bearing the software. As used herein, unless restricted to tangible “storage” media, terms such as computer or machine “readable medium” refer to any medium that participates in providing instructions to a processor for execution.

A machine-readable medium may take many forms, including but not limited to, a tangible storage medium, a carrier wave medium or physical transmission medium. Nonvolatile storage media include, for example, optical or magnetic disks, such as any of the storage devices in any computer(s), or the like, which may be used to implement the system or any of its components shown in the drawings. Volatile storage media may include dynamic memory, such as a main memory of such a computer platform. Tangible transmission media may include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media may include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASHEPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described herein may be embodied in a hardware device, it may also be implemented as a software only solution, for example, an installation on an existing server. In addition, data processing as disclosed herein may be implemented as a firmware, firmware/software combination, firmware/hardware combination, or a hardware/firmware/software combination.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-15. (canceled)

16. A method for early symptom detection and preventative healthcare, comprising:
receiving medical information related to a user;
receiving medical information related to individuals similar to the user;
implementing a risk predictive model using the medical information related to the user;
assessing a risk of a specific health outcome of the user using the risk predictive model; and
calibrating the risk predictive model using a decision control module by performing the steps of:
computing attribute scores for freshness, completeness and uncertainty of the medical information related to the user, wherein the attribute scores are computed using the medical information related to the user and the medical information related to the individuals similar to the user; and
assigning weights to the attribute scores;
computing a need-for-data (ND) score using the weighted attribute scores;
computing a need-for-checkup (NC) score using characteristics and traits of the user;
determining that new medical information related to the user is needed or that the user needs a checkup based on the ND score and the NC score; and
delivering a prompt to the user indicating that new medical information related to the user is needed or that the user needs a checkup.

17. The method of claim 16, wherein the medical information related to the user, the attribute scores, the ND score, and the NC score are stored in a database.

18. The method of claim 16, wherein the decision control module performs the further step of:
determining a usefulness parameter for the prompt by using a machine learning model on the medical information related to the user and on the medical information related to the individuals similar to the user.

19. The method of claim 16, wherein the decision control module performs the further step of:
determining a usefulness parameter for the prompt by using a deterministic algorithm on the medical information related to the user and on the medical information related to the individuals similar to the user.

20. The method of claim 18, further comprising:
comparing the ND score to a first threshold; and
comparing the usefulness parameter to a second threshold, wherein the prompt to the user indicating that new medical information related to the user is needed is delivered if the ND score is higher than the first threshold and the usefulness parameter is higher than the second threshold.

21. The method of claim 18, further comprising:
comparing the NC score to a first threshold; and
comparing the usefulness parameter to a second threshold, wherein the prompt to the user indicating that the user needs a checkup is delivered if the NC score is higher than the first threshold and the usefulness parameter is higher than the second threshold.

22. The method of claim 16, wherein the prompt to the user is delivered to a computing device of the user.

23. The method of claim 16, wherein the prompt to the user is delivered to a computing device of a relative or partner of the user.

24. The method of claim 16, wherein the weights of the attribute scores have magnitudes assigned using a machine learning model on the medical information related to the user and the medical information related to the individuals similar to the user.

25. The method of claim 16, wherein the medical information related to the user is obtained from a source selected from the group consisting of: a medical device, an electronic health record, a mobile phone, a wearable device, a biochemical test of the user, a genetic test of the user, a diary of the user, and a questionnaire answered by the user.

26. An electronic system for early symptom detection and preventative healthcare, comprising:
a database in which medical information related to a user is stored and in which medical information related to individuals similar to the user is stored; and
a processor that implements a risk predictive model and a decision control model, wherein the decision control model uses a machine learning model that is built on the database, and wherein the processor is configured for:
assessing a risk of a specific health outcome of the user using the risk predictive model based on the medical information related to the user and the medical information related to individuals similar to the user;
calibrating the risk predictive model by computing attribute scores for freshness, completeness and uncertainty of the medical information related to the user, and by assigning weights to the attribute scores;
computing a need-for-data (ND) score using the weighted attribute scores;
computing a need-for-checkup (NC) score using characteristics and traits of the user;
determining that new medical information related to the user is needed or that the user needs a checkup based on the ND score and the NC score; and
delivering a prompt to the user indicating that new medical information related to the user is needed or that the user needs a checkup.

27. The electronic system of claim 26, wherein the attribute scores for freshness, completeness and uncertainty are computed using the medical information related to the user and the medical information related to the individuals similar to the user.

28. The electronic system of claim 26, wherein the processor is further configured for:
determining a usefulness parameter for the prompt by using the machine learning model on the medical information related to the user and on the medical information related to the individuals similar to the user.

29. The electronic system of claim 26, wherein the processor is further configured for:
determining a usefulness parameter for the prompt by using a deterministic algorithm on the medical information related to the user and on the medical information related to the individuals similar to the user.

30. The electronic system of claim 28, wherein the processor is further configured for:
comparing the ND score to a first threshold; and
comparing the usefulness parameter to a second threshold, wherein the prompt to the user indicating that new medical information related to the user is needed is delivered if the ND score is higher than the first threshold and the usefulness parameter is higher than the second threshold.

31. The electronic system of claim 28, wherein the processor is further configured for:
comparing the NC score to a first threshold; and
comparing the usefulness parameter to a second threshold, wherein the prompt to the user indicating that the user needs a checkup is delivered if the NC score is higher than the first threshold and the usefulness parameter is higher than the second threshold.

32. The electronic system of claim 26, wherein the prompt to the user is delivered to a computing device of the user.

33. A method performed using a preventative healthcare system, comprising:
accessing from a database wellness information related to a user of the system, wherein the database contains the wellness information related to the user and characteristics and traits of the user;
implementing a risk predictive model based on the wellness information related to the user;
determining a probability of a specific health outcome of the user using the risk predictive model;
calibrating the risk predictive model by computing attribute scores for freshness, completeness and uncertainty of the wellness information related to the user, and by assigning weights to the attribute scores;
computing a need-for-data (ND) score using the weighted attribute scores;
computing a need-for-checkup (NC) score using the characteristics and traits of the user;
determining that new wellness information related to the user is needed or that the user needs a checkup based on the ND score and the NC score; and
delivering a prompt to the user indicating that new wellness information related to the user is needed or that the user needs a checkup.

34. The method of claim 33, further comprising:
determining a usefulness parameter for the prompt by using a machine learning model on the wellness information related to the user and on wellness information related to individuals similar to the user.

35. The method of claim 34, further comprising:
comparing the ND score to a first threshold; and
comparing the usefulness parameter to a second threshold, wherein the prompt to the user indicating that new wellness information related to the user is needed is delivered if the ND score is higher than the first threshold and the usefulness parameter is higher than the second threshold.
